(19) United States
(12) Patent Application Publication
(10) Pub. No.: US 2010/0063164 A1
(43) Pub. Date: Mar. 11, 2010

(54) TRANSPARENT ZINC SULPHIDE HAVING A HIGH SPECIFIC SURFACE AREA

(76) Inventors: Djamschid Amirzadeh-Asl, Moers (DE); Dirk Welp, Essen (DE); Jörg Hocken, Meerbusch (DE); Jochen Winkler, Rheurdt (DE)

Correspondence Address: FULBRIGHT & JAWORSKI, LLP, 666 FIFTH AVE, NEW YORK, NY, 10103-3198, US

(21) Appl. No.: 12/515,973

(22) PCT Filed: Dec. 3, 2007
(86) PCT No.: PCT/EP07/63199

§ 371 Date: Aug. 3, 2009

(30) Foreign Application Priority Data
Jan. 12, 2006 (DE) 10-2006-057-224.6

(51) Int. Cl. A61K 47/02; C07F 3/06; C08K 3/30; C09J 1/00; C04B 28/00; A61K 8/27

(52) U.S. Cl. 514/769; 556/118; 524/420; 106/287.18; 106/815

(57) ABSTRACT

Zinc sulphide having a high specific surface area, a process for producing it and the use of this zinc sulphide.

The present invention provides a transparent zinc sulfide having a high specific surface area, a process for the production thereof and the use of this zinc sulfide.

Company brochure no. 1119596 from Sachtleben GmbH, Duisburg, Germany, discloses a zinc sulfide pigment, in the production of which highly purified solutions of zinc salt and sodium sulfide are used. The high reflectance of the 300 nm particles in visible light and the near UV range gives a neutral white shade and optimum scattering, hiding and brightening power when used in coatings and plastics. Owing to its white pigment properties, zinc sulfide is used where organic or inorganic binders have to be highly pigmented for specific applications, e.g. in undercoats, jointing and sealing compounds, primers etc. Plastics are also pigmented with zinc sulfide, for example melamine, urea and polyester moulding compositions, providing these with excellent colouring properties. In addition, other properties, such as e.g. increased flame resistance, are achieved.

Owing to its relatively low Mohs hardness of 3 and its spherical particle shape, zinc sulfide has very low abrasiveness and therefore does not cause any metal abrasion during processing. Zinc sulfide is used on a large scale as a white pigment, particularly in fibre-reinforced plastics since titanium dioxide, which is employed as an alternative white pigment, leads to breakage of the glass fibres owing to its higher Mohs hardness of 5.5 to 6.5, unlike zinc sulfide.

EP-B-1463411 discloses the use of zinc sulfide as an agent against mites in threads, fibres and filaments. In addition, the use of zinc sulfide in a liquid or solid composition for the cleaning and/or treatment of textile surfaces is disclosed.

DE-A-10051578 discloses a process for the production of yarns, fibres or filaments with significantly better whiteness and lower yellowing. The process comprises the mixing of a masterbatch of zinc sulfide and polyester in the melt and subsequent spinning from the melt. In this process, the zinc sulfide is used as a white pigment in a proportion of 0.1 to 3 wt. % to delustre the polyester fibre products.

The typical particle size of the zinc sulfides used above as a white pigment is approximately 300 nm, its specific surface area (BET) is 2 to 10 m²/g and it displays a relative brightening power of approx. 380 (DIN 55982).

Because of these properties, this zinc sulfide is not transparent in the visible range. Up to the present, therefore, it has not been suitable as an additive for applications in which transparency or colour consistency are desired, so that up to now it has been necessary to have recourse to other materials. However, these do not possess the low Mohs hardness and biocidal properties of zinc sulfide, e.g. against mites.

The object of the invention is to provide a zinc sulfide which, on the one hand, is transparent, i.e. in which the white pigment properties are reduced or completely lacking, and on the other hand possesses the desired low Mohs hardness and desired biocidal properties.

Surprisingly, the object is achieved by the zinc sulfide according to the invention.

In particular, this object is achieved by an extremely fine-particle, i.e. nanoscale, zinc sulfide with an average crystallite size of less than 250 nm, preferably less than 150 nm, particularly preferably less than 80 nm, most particularly preferably less than 40 nm.

The zinc sulfide according to the invention possesses an extremely low scattering and brightening power with a value of less than 300 (DIN 55982), preferably less than 100, particularly preferably less than 70, so that no hiding power is achieved when incorporated e.g. into mouldings and coatings. The specific surface area (BET, determined in accordance with DIN-ISO 9277) is 15 to 300 m²/g, preferably 30 to 250 m²/g, particularly preferably 50 to 200 m²/g.

The zinc sulfide according to the invention lacks or has reduced white pigment properties. It has a low Mohs hardness and has a biocidal effect, particularly against algae, fungi and bacteria.

The zinc sulfide according to the invention is produced by bringing together a compound containing sulfide sulfur with a solution containing a zinc compound, as a result of which zinc sulfide is precipitated as a solid. This solid is optionally isolated by washing, filtration and subsequent drying.

The zinc sulfide according to the invention is produced for example in that the aqueous solution of a compound containing sulfide sulfur is mixed, in a suitable concentration and at a suitable temperature, with an aqueous solution containing a zinc compound, controlling the mixing so that a specific pH, preferably a pH of 5, particularly preferably of 3 to 4, is not exceeded, and after precipitation of the zinc sulfide the pH of the suspension is adjusted to a value of about 7 by further addition of aqueous solution of the compound containing sulfide sulfur while stirring, the zinc sulfide obtained is filtered, washed until the required freedom from salt is achieved, dried and optionally ground.

Metal sulfides and/or metal polysulfides, for example, are used as compounds containing sulfide sulfur, preferably those from the alkali group. According to the invention, it is also possible to use gaseous hydrogen sulfide (H₂S), in which case the H₂S is introduced into the solution of the zinc compound. Alternatively, it is also possible to use organic sulfide sulfur carriers, e.g. thioacetamide, for the production of the zinc sulfide according to the invention. Mixtures of compounds containing sulfide sulfur are also possible.

Zinc sulfate and/or zinc chloride and/or an organozinc compound, for example zinc acetate, are preferably used as zinc compounds for the precipitation of extremely fine particle zinc sulfide. Mixtures of these zinc compounds are also possible.

If several starting solutions are used to precipitate the zinc sulfide according to the invention, the solutions can be added in any combination and in any order.

According to the invention, it is possible to use any process known from the prior art for the precipitation of the extremely fine particle zinc sulfide, e.g. precipitation in a precipitation cell, in a T/Y positive mixer, in a micro-reactor or in a micro-jet reactor, by either a continuous or a batchwise operating method in each case.

The precipitation can take place both in one step and in multiple steps, preferably in two steps.

The primary particle size can be controlled e.g. by varying the feedstock solutions or their concentrations, varying the temperature or varying the residence times.

Furthermore, carrying out a precipitation in an autoclave results in a wide variety of process parameter combinations by means of which the desired particle size can be adjusted.

The zinc sulfide suspension thus obtained is then worked up by processes from the prior art to form the finished product. The suspension is then usually filtered and, depending on the product requirements, washed until salt-free, dried and, if necessary, ground.

The drying can take place for example in a rotary tubular furnace, a spray dryer or a hearth furnace, but also by freeze-drying. The removal of water by the technique of flushing is also possible.

The dried product can be micronised e.g. in a pin mill, a Coloplex mill, a Zircoplex mill, a steam mill or an air jet mill, depending on the application.

The zinc sulfide according to the invention can be prepared for the various applications, after working up the precipitation suspension according to the prior art, as fine particle size suspensions, as a slurry, as a paste or as a powder after drying and optionally grinding.

The primary crystallite size of the zinc sulfide according to the invention is less than 250 nm, preferably less than 150 nm, particularly preferably less than 80 nm, most particularly preferably less than 40 nm.

Since the zinc sulfide nanoparticles according to the invention exhibit very highly modified and new product properties, it is particularly preferred according to the invention that the individual particles are not present in agglomerated form so that they are optimally distributed during processing for the various applications. According to the invention, therefore, the zinc sulfide according to the invention can be post-treated inorganically and/or organically, as is conventional for example with the known titanium dioxide pigments and as described for example in the following documents: EP 1 576 061 A2, U.S. Pat. No. 4,052,224 A1, U.S. Pat. No. 3,941,603 A1 and U.S. Pat. No. 4,075,031 A1. The inorganic post-treatment of the zinc sulfide according to the invention can take place according to the invention in a similar manner. The inorganic post-treatment of the zinc sulfide preferably takes place by means of post-treatment reagents such as SiO₂, Al₂O₃, ZrO₂, TiO₂ and/or metal phosphates.

The inorganic post-treatment preferably takes place before the drying of the zinc sulfide according to the invention. For this purpose, the zinc sulfide filter cake is re-dispersed in an aqueous medium and then post-treated by adding one or more of the above-mentioned post-treatment reagents. The post-treatment takes place according to the prior art relating to the inorganic post-treatment of pigments. The subsequent work-up takes place as already set out above.

The inorganic surface modification of the ultrafine zinc sulfide according to the invention can consist of compounds which contain the following elements: aluminium, antimony, barium, calcium, cerium, chlorine, cobalt, iron, phosphorus, carbon, manganese, oxygen, sulfur, silicon, nitrogen, strontium, vanadium, tin and/or zirconium compounds or salts. Sodium silicate, sodium aluminate and aluminium sulfate can be mentioned as examples.

The inorganic surface treatment of the ultrafine zinc sulfide according to the invention takes place for example in an aqueous slurry. The reaction temperature in this case should preferably not exceed 50° C. The pH of the suspension is adjusted to pH values in the range of more than 9, e.g. using NaOH. While stirring vigorously, the post-treatment chemicals (inorganic compounds), preferably water-soluble inorganic compounds such as e.g. aluminium, antimony, barium, calcium, cerium, chlorine, cobalt, iron, phosphorus, carbon, manganese, oxygen, sulfur, silicon, nitrogen, strontium, vanadium, tin and/or zirconium compounds or salts, are then added. The pH value and the quantities of post-treatment chemicals are selected according to the invention so that the latter are completely dissolved in water. The suspension is stirred intensively so that the post-treatment chemicals are homogeneously distributed in the suspension, preferably for at least 5 minutes. In the next step, the pH of the suspension is reduced. It has proved advantageous here for the pH to be reduced slowly while stirring vigorously. Particularly advantageously, the pH is reduced within 10 to 90 minutes to values of 5 to 8. This is then followed according to the invention by a maturing time, preferably a maturing time of about one hour. The temperatures should preferably not exceed 50° C. during this time. The aqueous suspension is then washed and dried. To dry the ultrafine, surface-modified zinc sulfide according to the invention, spray drying, freeze drying and/or attrition drying are suitable. Depending on the drying process, subsequent grinding of the dried powder may be necessary. The grinding can be carried out by processes which are known per se.

The organic post-treatment can take place before or after drying, tempering and/or grinding. It is also possible according to the invention to post-treat the zinc sulfide according to the invention organically if it is in the form of a paste or suspension.

According to the invention, the following compounds are suitable as organic surface modifiers: polyethers, silanes, polysiloxanes, polycarboxylic acids, fatty acids, polyethylene glycols, polyesters, polyamides, polyalcohols, organic phosphonic acids, titanates, zirconates, alkyl and/or aryl sulfonates, alkyl and/or aryl sulfates, alkyl and/or aryl phosphoric acid esters.

The production of organically surface-modified zinc sulfide according to the invention can take place according to processes which are known per se.

On the one hand these comprise surface modification in an aqueous or solvent-containing phase. On the other hand, the organic component can be applied on to the particle surface by direct spraying followed by mixing/grinding.

According to the invention, suitable organic compounds are added to a zinc sulfide suspension while stirring vigorously and/or while dispersing. In this case the organic modifications are bound to the particle surface by chemisorption/physisorption.

Suitable examples of organic compounds are compounds selected from the group of the alkyl and/or aryl sulfonates, alkyl and/or aryl sulfates, alkyl and/or aryl phosphoric acid esters or mixtures of at least two of these compounds, wherein the alkyl or aryl radicals can be substituted by functional groups. The organic compounds can also be fatty acids which optionally possess functional groups. Mixtures of at least two of these compounds can also be used.

The following are used, for example: alkylsulfonic acid salt, sodium polyvinyl sulfonate, sodium-N-alkylbenzene sulfonate, sodium polystyrene sulfonate, sodium dodecylbenzene sulfonate, sodium lauryl sulfate, sodium cetyl sulfate, hydroxylamine sulfate, triethanolammonium lauryl sulfate, phosphoric acid monoethyl monobenzyl ester, lithium perfluorooctane sulfonate, 12-bromo-1-dodecane sulfonic acid, sodium 10-hydroxy-1-decane sulfonate, sodium carrageenan, sodium 10-mercapto-1-cetane sulfonate, sodium 16-cetene(1) sulfate, oleyl cetyl alcohol sulfate, oleic acid sulfate, 9,10-dihydroxystearic acid, isostearic acid, stearic acid, oleic acid.

The organic additive is preferably selected from: carboxylic acids, soaps, metal soaps, alcohols (e.g. 1,1,1-trimethylolpropane), pentaerythritol, neopentyl glycol, polyglycols (e.g. polyethylene glycol), polyethylene glycol ethers, organic esters (e.g. neopentyl glycol dibenzoate), silanes, siloxanes, silicone oils, organic sulfones with the formula RSO₂R, organic ketones (R—(C═O)—R), organic nitriles (RCN), organic sulfoxides (R₂—SO₂), organic amides (R—(C═O)—NR′R or R—(S═O)—ONR′R), fatty acid esters, fatty acid amides or mixtures of two or more of these substances. R and R′ here denote saturated or unsaturated hydrocarbons, such as e.g. alkyl (—CH2-CH2-)ₙ, cyclic compounds or organometallic compounds. R and R′ can be the same or different here.

The zinc sulfide according to the invention can be used e.g. in: plastics, particularly in polymer production (e.g. of thermoplastic or thermosetting polymers), varnishes, paints, fibres, paper (e.g. laminated paper), adhesives, ceramics (e.g. electroceramics and magnetic ceramics), enamel, adsorbing agents, ion exchangers, grinding and polishing agents, cutting fluids and cutting fluid concentrates, fire-resistant products, hard concrete materials, medicinal products and cosmetics (e.g. powders, ointments, toothpaste).

The zinc sulfide according to the invention can be used particularly in applications in which white pigment properties are considered undesirable but nevertheless the properties of zinc sulfide have an advantageous effect on the system. These are for example synthetic organic polymers, mouldings produced therefrom, coatings such as varnishes and paints and/or jointing and sealing compounds with good transparency and/or colour.

Preferably 0.01 to 55 vol. %, particularly preferably 0.1 to 45 vol. % of the zinc sulfide according to the invention is used, based in each case on the finished product.

Synthetic organic polymers are understood to be all thermosets, elastomers and thermoplastics, which can also contain other processing aids such as stabilisers, plasticisers, organic and/or inorganic pigments, dyes, glass fibres and/or other additives.

The zinc sulfide according to the invention can also be used in applications in which an improvement of the properties are desired without the pigmentary nature of the zinc sulfide being relevant.

When 0.1 to 30 wt. %, preferably 0.2 to 15 wt. %, particularly preferably 0.3 to 10 wt. %, of the zinc sulfide according to the invention is used in elastomers, heat stabilisation can be achieved, particularly in combination with organic stabilisers such as alkylidene bisphenols.

The zinc sulfide according to the invention can be used in thermoplastics as a heavy metal deactivator. The quantity of zinc sulfide to be added here is 0.1 to 30 wt. %, preferably 0.2 to 15 wt. %, particularly preferably 0.5 to 10 wt. %, based on the quantity of the thermoplastic. In this case, a further addition of organic complexing agents in the thermoplastics can be omitted.

The use of the ZnS according to the invention can bring about improvements in the mechanical properties of thermosets and thermoplastic polymers, such as e.g. hardness, flexural strength, impact resistance etc.

In addition, the zinc sulfide according to the invention can be used as a friction additive, e.g. in lubricants, brake linings or clutches etc.

The evaluations of all the listed non-pigmentary properties of the zinc sulfide according to the invention are equivalent to or significantly more advantageous than the corresponding properties of the significantly coarser zinc sulfide according to the prior art.

When the zinc sulfide according to the invention is used in coatings, these display a biocidal action, e.g. against algae, bacteria or moulds. The quantity of zinc sulfide added in this case is 0.1 to 30 wt. %, preferably 0.2 to 15 wt. %, particularly preferably 0.5 to 10 wt. %, based on the quantity of the coating material.

In addition, the zinc sulfide according to the invention can also be used as a catalyst.

The invention is explained in more detail by the following example, without being limited thereto:

### EXAMPLE 1

50 ml of distilled water at a temperature of 65° C. are initially charged into a receiving vessel and 500 ml of an aqueous ZnSO₄ solution (120 g/l) and 500 ml of an aqueous Na₂S solution (60 g/l) are added simultaneously with stirring. The solutions are also at a temperature of 65° C. The addition of the two solutions into the receiving vessel is controlled so that the pH value in the suspension is 3 to 4.

Once precipitation of the zinc sulfide has taken place, the pH of the suspension is adjusted to 7 to 7.5 by adding more of the Na₂S solution, while stirring. Once the suspension has been filtered and washed several times until a conductivity of less than 100 μS is achieved, the zinc sulfide is dried at 120 to 150° C.

The zinc sulfide produced in this way is crystalline and has an average particle size of 5 nm. The surface area is 160 m²/g (BET) and the brightening power approx. 30 (DIN 55982).

1-25. (canceled)

26. Zinc sulfide lacking or having reduced white pigment properties and that is transparent.

27. Zinc sulfide according to claim 26, having low scattering and brightening power with a value of less than 300 in accordance with DIN 55982.

28. Zinc sulfide according to claim 26, having a specific surface area of 15 to 300 m²/g according to a BET method determined in accordance with DIN-ISO 9277).

29. Zinc sulfide according to claim 26, wherein it is nanoscale.

30. Zinc sulfide according to claim 26, having an average crystallite size of less than 250 nm.

31. Zinc sulfide according to claim 26, wherein zinc sulfide crystallites in the zinc sulfide are inorganically pot treated, organically post-treated or both inorganically and organically post-treated.

32. A process comprising preparing the zinc sulfide of claim 26 by:
mixing an aqueous solution of a compound containing sulfide sulfur in a suitable concentration and at a suitable temperature with an aqueous solution containing a zinc compound;
controlling the mixing so that a specific pH is not exceeded to precipitate zinc sulfide;
adjusting the pH of the suspension to about 7 by addition of the aqueous solution of the compound containing sulfide sulfur while stirring;
filtering the zinc sulfide obtained;
washing the filtered zinc sulfide until the required freedom from salt is achieved; and
drying the zinc sulfide.

33. A process according to claim 32, wherein the primary particle size of the zinc sulfide is controlled by selection of the feedstock solutions.

34. A process according to claim 32, wherein the compound containing sulfide sulfur comprises at least one of a metal sulfide, a metal polysulfide, or an organic sulfide sulfur carrier.

35. A process according to claim 34, wherein the compound containing sulfide sulfur comprises an alkali group metal or is thioacetamide.

36. A process according to claim 32 the zinc compound is selected from the group consisting of zinc sulfate, zinc chloride or an organozinc compound.

37. A process for the production of zinc sulfide according to claim 26, comprising the steps of:
introducing gaseous hydrogen sulfide into aqueous solution of a zinc compound, wherein addition of the gaseous hydrogen sulfide is controlled so that a specific pH is not exceeded, to precipitate zinc sulfide in a suspension;
adjusting the pH of the suspension to a value of about 7 by further addition of the gaseous hydrogen;
filtering the zinc sulfide;
wash the filtered zinc sulfide until the required freedom from salt is achieved; and
drying the washed zinc sulfide.

38. A process according to claim 32, wherein the precipitation of the extremely fine particle zinc sulfide takes place in a precipitation cell, a T/Y positive mixer, a micro-reactor or a micro-jet reactor, by a continuous or batchwise operating method.

39. A process according to claim 37, wherein the precipitation of the extremely fine particle zinc sulfide takes place in a precipitation cell, a T/Y positive mixer, a micro-reactor or a micro jet reactor, by a continuous or batchwise operating method.

40. A process according to claim 32, wherein the precipitation can take place both in one step or in multiple steps.

41. A process according to claim 37, wherein the precipitation can take place both in one step or in multiple steps.

42. A process according to claim 32, wherein the drying takes place in a rotary tubular furnace, a spray dryer, a hearth furnace or by freeze-drying.

43. A process according to claim 37, wherein the drying takes place in a rotary tubular furnace, a spray dryer, a hearth furnace or by freeze-drying.

44. A process according to claim 32, wherein water removal is conducted via flushing.

45. A process according to claim 37, wherein water removal is conducted via flushing.

46. A process according to claim 32, wherein the zinc sulfide is micronized in a pin mill, a Coloplex mill, a Zircoplex mill, a steam mill or an air jet mill.

47. A process according to claim 37, wherein the zinc sulfide is micronized in a pin mill, a Coloplex mill, a Zircoplex mill, a steam mill or an air jet mill.

48. A synthetic, organic polymer, a molding, a coating, a jointing compound or a sealing compound comprising the zinc sulfide of claim 26.

49. A plastic, a varnish, a paint, a fiber, a paper, an adhesive, a ceramic, an enamel, an adsorbing agent, an ion exchanger, a grinding agent, a polishing agent, a cutting fluid, a cutting fluid concentrate, a fire-resistant product, a hard concrete material, a medicinal product or a cosmetic comprising the zinc sulfide of claim 26 and a suitable carrier.

50. A method of improving the mechanical properties of a thermoset or a thermoplastic polymer comprising adding a sufficient amount of the zinc sulfide of claim 26 to the thermoset or thermoplastic polymer.

51. A elastomer comprising a sufficient amount of the zinc sulfide of claim 26 to stabilize the elastomer against heat.

52. A thermoplastic comprising a sufficed amount of the zinc sulfide of claim 26 to deactivate a heavy metal.

53. A lubricant, a brake lining or a clutch comprising the zinc sulfide of claim 26.

54. A biocide comprising the zinc sulfide of claim 26.

55. A catalyst comprising the zinc sulfide of claim 26.

56. A fine-particle suspension, a slurry, a paste or a powder comprising the zinc sulfide of claim 26.
